(19) United States
(12) Patent Application Publication
(10) Pub. No.: US 2005/0107857 A1
(43) Pub. Date: May 19, 2005

(54) Custom heater for hot compress pack

(76) Inventor: Chin-Shen Liang, Rende Hsiang (TW)

Correspondence Address: BACON & THOMAS, PLLC, 625 SLATERS LANE, FOURTH FLOOR, ALEXANDRIA, VA, 22314, US

(21) Appl. No.: 10/986,302

(22) Filed: Nov. 12, 2004

(30) Foreign Application Priority Data
Nov. 17, 2003 (TW) 092220287

(51) Int. Cl. A61F 7/00

(52) U.S. Cl. 607/114

(57) ABSTRACT

A custom heater for a hot compress pack includes a base, a temperature sensor, a lid and a power cord. The base includes a heating module configured in the base. The heating module includes a heat transfer wire of a heat conductor and a circuit board. The circuit board is connected to the heat transfer wire of the heat conductor by an electric wire. The temperature sensor switch is connected to the circuit board of the heating module by the electric wire. An inner part of the lid can be pivoted on the base. The power cord is connected with the circuit board.

### BACKGROUND OF THE INVENTION

1. Field of the Invention

The inventions relates in general to a custom heater for a hot compress pack, and more particularly to a heater utensil that can heat a hot compress pack, so as to achieve a structure design of convenience and safety.

2. Description of the Related Art

A hot compress pack can provide comfort to a part of a body, so as to achieve an effect of promoting blood circulation and fatigue relief. The conventional hot compress pack available in the market is mainly filled with fluid gel. The gel includes a high efficacy to accumulate heat. Besides, the conventional hot compress pack can be put in a microwave oven or hot water to for use of the hot compress pack. The conventional hot compress pack features in heat accumulation and can prolong an elapsed time of cooling, so as to extend the elapsed time for hot compress remedy. However, the conventional compress pack has a disadvantage that the heating elapsed time of approximately 30 to 50 seconds depends on a size of the gel. Since the heating elapsed time is very short, the hot compress pack body may spoil due to being overheated, whereby it becomes unusable or very inconvenient to clean the spoilt hot compress pack.

Further, microwave ovens are not found in very home and so it is rather inconvenient for those people to heat the compress pack. The alternative to using the microwave oven, i.e., putting the hot compress pack into hot water, may cause danger through scalding, as well as making a treatment space wet through spillage. Thus, there is a need to overcome the present drawbacks of heating a hot compress pack.

### SUMMARY OF THE INVENTION

An objective of the present invention is to provide a custom heater for a hot compress pack, which is safe for use and includes a high level of warmth retention. Thereby heating the hot compress pack is much more convenient and safe.

In order to achieve the above-mentioned objective of structural features and techniques, a custom heater for a hot compress pack of the present invention is provided. The custom heater for the hot compress pack mainly includes a base, a temperature sensor, a lid and a power cord. The base includes a heating module configured in the base. The heating module includes a heat conductor and a circuit board, and the circuit board is connected to the heat conductor by an electric wire. The temperature sensor switch is connected to the circuit board of the heating module by an electric wire. The lid is preferably hinged to the base. The power cord is connected with the circuit board. With such a design, the hot compress pack can be heated on the custom heater, so as to achieve a superior effectiveness in both convenience and safety.

### BRIEF DESCRIPTION OF THE DRAWINGS

FIG. 1 is a perspective view of a custom heater for a hot compress pack in accordance with the present invention.

FIG. 2 is a perspective view of the custom heater in FIG. 1, in an opened status.

FIG. 3 is a perspective view of the custom heater in FIG. 1, in a closed status.

### DETAILED DESCRIPTION OF THE INVENTION

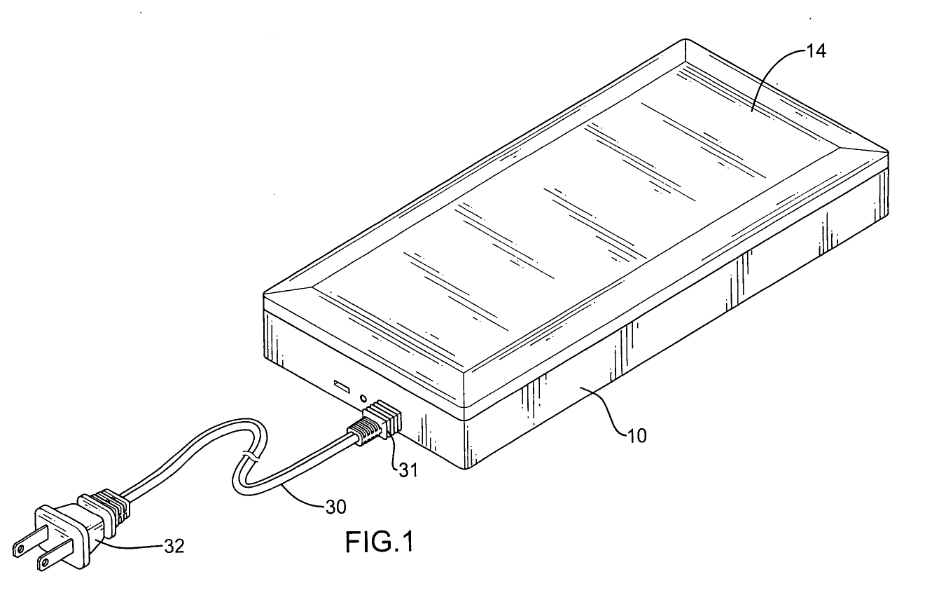

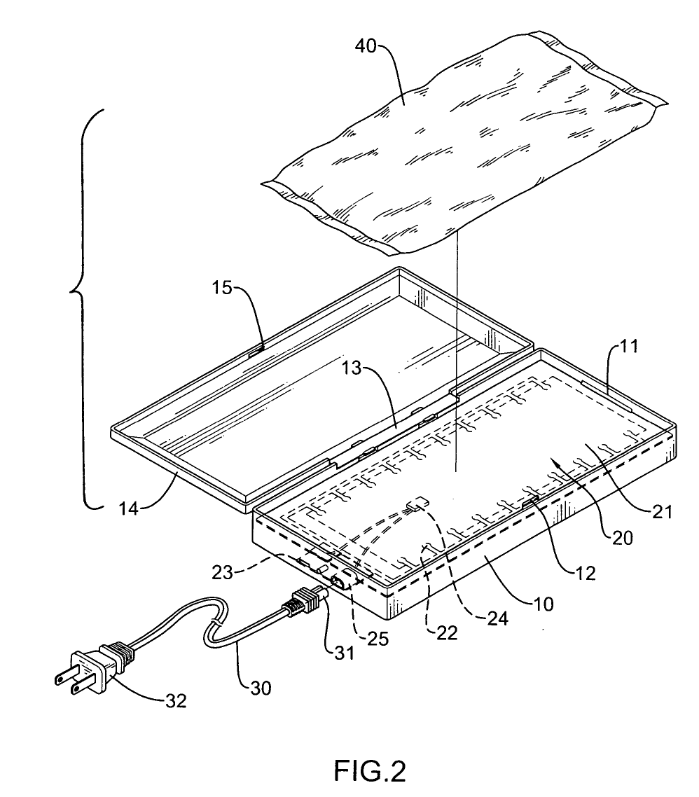

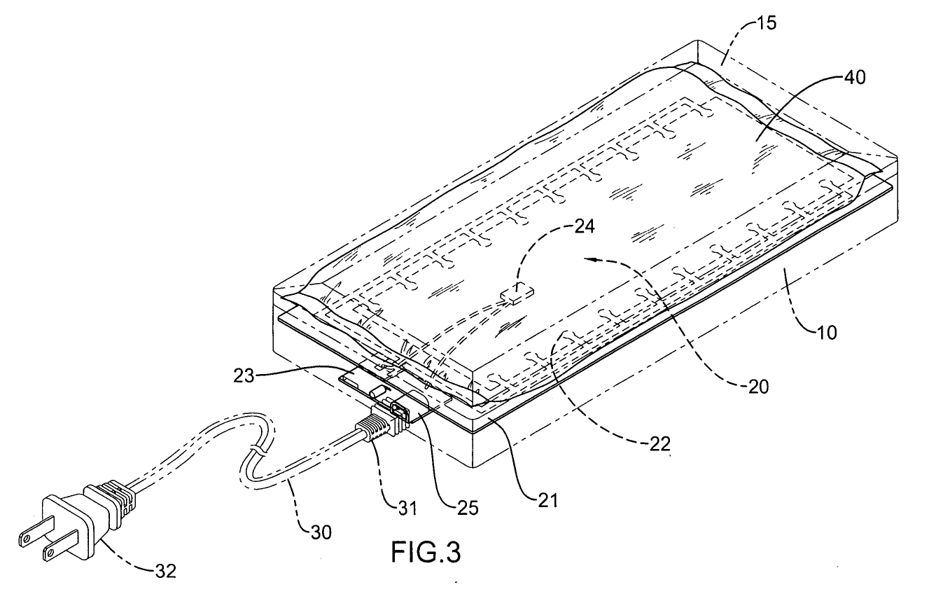

Referring to FIG. 1, FIG. 2 and FIG. 3, a custom heater for a hot compress pack includes a base 10, a temperature sensor 24, a lid 14 and a power cord 30. The base 10 includes a heating module 20 received in the base. The heating module 20 includes a heat conductor 21 and a circuit board 23. The heat conductor 21 can be a heating pipe or a plate with heat transfer wires 22 of screen-printing.

The base 10 has a floor from which a peripheral wall extends to form a recess with a top opening, and a plurality of pillars extend upward from the floor to support the heat conductor 21 and to allow heated air to circulate freely underneath the heat conductor 21. A plurality of buckling parts 11 are configured on an inner part of the base 10 to secure the heat conductor 21 to the base 10. The circuit board 23 is connected to the heat transfer wire 22 of the heat conductor 21 by an electric wire. The temperature sensor switch 24 is connected to the circuit board 23 of the heating module 20 by the electric wire. Moreover, the lid 14 can be pivoted on the base with a hinge 13, and an external part of the lid 14 is configured with a slit 15 for engaging with a catch on the base 10. One end of the power cord 30 is installed with a connector 31, and the other end of the power cord 30 is installed with a plug 32. The connector 31 can be connected to a socket 25 of the circuit board 23. To heat the hot compress pack 40, it can be put inside the base 10 and covered with the lid 14. Then the plug 32 is plugged in the socket of a power source, so that an electric current can go through the circuit board 23 and the heat transfer wire 22 to heat the heat conductor 21. Thereby the hot compress pack 40 placed on top of the heat conductor 21 and covered with the lid can be efficiently heated. The temperature sensor switch 24 can disconnect the electric current of the heat transfer wire 22 to stop heating the heat conductor 21 when sensing the temperature has reached an upper limit. At this moment, the heat compress pack 40, which has accumulated the heat, can be taken out for use. If the heat compress pack 40 remains in the custom heater, to minimize any cooling down.

In conclusion, the present invention for a custom heater for a hot compress pack can dispense with the prior art heating in a microwave oven or hot water, whereby a practical and safer effect to heat and keep warm a hot compress pack is achieved. Therefore, the present invention exactly comprises utility and conforms to novelty non-obviousness and inventive step of requirements of a patent.

While the invention has been described by way of example and in terms of a preferred embodiment, it is to be understood that the invention is not limited thereto. On the contrary, it is intended to cover various modifications and similar arrangements and procedures, and the scope of the appended claims therefore should be accorded the broadest interpretation so as to encompass all such modifications and similar arrangements and procedures.

1. A custom heater for a hot compress pack comprising:
a base comprising a heating module configured in the base, wherein the heating module comprises a heat conductor and a circuit board, and wherein the circuit board is connected to the heat conductor by an electric wire;
a temperature sensor switch connected by an electric wire to the circuit board of the heating module;
a lid securable on the base; and
a power cord connected with the circuit board.

2. The custom heater for a hot compress pack as claimed in claim 1, wherein the base has a floor from which a peripheral wall extends upward to define a recess in the base, and a plurality of pillars are formed on the floor of the base to support the heat conductor, and wherein securing means is fitted on an inner part of the base to secure the heat conductor to the base.

3. The custom heater for a hot compress pack as claimed in claim 1, wherein the lid is hinged to the base.

4. The custom heater for a hot compress pack as claimed in claim 2, wherein the lid is hinged to the peripheral wall of the base.

5. The custom heater for a hot compress pack as claimed in claim 3, wherein a first end of the power cord is installed with a connector, and a second end of the power cord is installed with a plug, and wherein the connector can be connected to a socket of the circuit board and the plug is receivable in a mains electricity outlet.

6. The custom heater for a hot compress pack as claimed in claim 4, wherein a first end of the power cord is installed with a connector, and a second end of the power cord is installed with a plug, and wherein the connector can be connected to a socket of the circuit board and the plug is receivable in a mains electricity outlet.

7. The custom heater a the hot compress pack as claimed in claim 5, wherein the heat conductor of the heating module is a plate with heat transfer wires of screen printing.

8. The custom heater for the hot compress pack as claimed in claim 6, wherein the heat conductor of the heating module is a plate with heat transfer wires of screen printing.

9. The custom heater for the hot compress pack as claimed in claim 5, wherein the heating module is a heating pipe.

10. The custom heater for the hot compress pack as claimed in claim 6, wherein the heating module is a heating pipe.
